(19) United States
(12) Patent Application Publication
(10) Pub. No.: US 2014/0046348 A1
(43) Pub. Date: Feb. 13, 2014

(54) WOUND HEALING SYSTEM

(71) Applicant: Hooman Soltanian, Chagrin Falls, OH (US)

(72) Inventor: Hooman Soltanian, Chagrin Falls, OH (US)

(73) Assignee: UNIVERSITY HOSPITALS OF CLEVELAND, Cleveland, OH (US)

(21) Appl. No.: 13/961,056

(22) Filed: Aug. 7, 2013

Related U.S. Application Data
(60) Provisional application No. 61/680,452, filed on Aug. 7, 2012.

(51) Int. Cl. A61B 17/064

(52) U.S. Cl. CPC A61B 17/064
USPC 606/151

(57) ABSTRACT

A novel system for securing a material to tissue is disclosed. The system may include a body and a plurality of engagement members extending from the body. The engagement members may be adapted to at least partially traverse the material, and each engagement member may include a support at least partially traversing the material and at least one securing member extending from the support and adapted to engage the tissue. The system may be useful for facilitating wound healing by maintaining contact between tissue and a material, such as a skin graft, during healing.

### CROSS-REFERENCE TO RELATED APPLICATIONS

This application claims priority to U.S. Provisional Patent Application Ser. No. 61/680,452, filed on Aug. 7, 2012. The contents of that application are fully incorporated by reference herein.

### BACKGROUND

The present disclosure relates generally to devices and methods for wound healing, and more specifically to devices and methods for maintaining compressive contact between tissue and wound healing material to facilitate wound healing.

Wound healing often involves the use of grafts or similar materials being placed into contact with a wound. One common application is the use of a skin graft to facilitate wound healing. Skin grafts often require multiple days of immobilization to the recipient tissue in order to allow ingrowth of new blood vessels and vascularization of the graft. To immobilize the graft, suture ties or staples have been used. The use of sutures is time consuming and not always successful, especially for large areas of skin graft. The sutures may also require removal. If staples are used, they may also need to be removed later. In addition, dressing changes are typically required and the process of changing a graft dressing is typically painful for the patient.

Recently, negative pressure dressings have become increasingly popular for skin grafts. Negative pressure dressings are expensive and difficult to use in certain areas of the body (e.g., groin, perineum, axilla, and face). In addition, negative pressure dressings require the attachment of the patient to a vacuum pump for the period of time, which is cumbersome and expensive.

### BRIEF SUMMARY

It is desirable to provide systems for facilitating wound healing, such as with a graft. The present disclosure includes novel wound healing systems, methods for making such systems, and methods for using such systems.

According to an aspect of the disclosure, there is provided an engagement member for securing a material to tissue. The engagement member may include a support adapted to at least partially traverse the material; a plurality of securing members extending from the support at a plurality of locations along the length of the support, the securing members being adapted to engage the tissue; and a retaining member operatively coupled to the support and adapted to prevent the entire engagement member from traversing the material.

The securing members may be adapted to engage tissue that is at least one of: synthetic, biologic, or combinations thereof. A first of the plurality of locations may be farther from the retaining member than a second of the plurality of locations. Also, the support may be adapted to be trimmed between two of the plurality of securing members by standard surgical scissors such that the length of the support is shortened and at least one of the plurality of securing members is removed from the support.

The plurality of securing members may also have a plurality of lengths. In addition, the plurality of locations along the length of the support may be distributed vertically and laterally on the support. Moreover, the support and the retaining member may be integrally formed. Also, the support and the plurality of securing members may be integrally formed.

The retaining member may also be adjustable such that adjustment of the retaining member changes the distances between the retaining member and the plurality of locations. For example, the support may include external threads and the retaining member may include internal threads.

In addition, at least one of the plurality of securing members may extend from the support such that at least part of the securing member is closer to the retaining member than the end of the securing member extending from the support is to the retaining member. For example, at least one of the securing members may be curved. Also, at least one of the securing members may extend from the support at an angle of from about 30 degrees to about 60 degrees.

The engagement member may also be made at least in part from bioabsorbable material. The engagement member may also be adapted to facilitate delivery of wound healing factors to at least one of the tissue or the material. The wound healing factors may include, for example, at least one of: antibiotics, growth factors, interleukin, topical antibacterial materials, anti-tumor agents, and absorbent materials to control the wound drainage.

The engagement member may also be adapted to facilitate compression of the material and tissue, and wherein the compression facilitates vascularization of the material. In addition, multiple engagement members may be used, and the engagement members may have supports of different lengths. Also, the system may include a connector joining multiple engagement members.

According to another aspect of the disclosure, there is provided a system for securing a material to tissue. The system may include a body; and a plurality of engagement members extending from the body, the engagement members being adapted to at least partially traverse the material. Each of the engagement members may include a support at least partially traversing the material; at least one securing member extending from the support and adapted to engage the tissue.

The body may include pores to facilitate drainage from the tissue. The body may also include a pliable sheet. At least part of the system, such as the body or the engagement members, may include wound healing factors. Also, the material may be a graft. The plurality of supports may also have a plurality of lengths. The support and the body may be integrally formed.

The engagement members and the body may be adapted to facilitate compression of the material and tissue, and wherein the compression facilitates vascularization of the material. The engaging member may further include a retaining member adapted to prevent the engagement member from completely traversing the material. In addition, the securing members and the retaining members may be on opposite sides of the body. The retaining member may also be adjustable such that adjustment of the retaining member changes the distances between the retaining member and the plurality of locations. For example, the support may include external threads and the retaining member may include internal threads.

According to another aspect of the disclosure, there is provided a method for compressing a material and a tissue to facilitate wound healing. The method may include placing a plurality of engagement members at least partially through the material. The engagement members may include a support at least partially traversing the material, at least one securing member extending from the support and adapted to engage the tissue, and a retaining member adapted to prevent the engagement member from completely traversing the material. The method may further include urging the plurality of engagement members into the tissue such that the securing members engage the tissue and prevent separation of the tissue and the material.

According to another aspect of the disclosure, there is provided another method for compressing a material and a tissue to facilitate wound healing. The method may include placing a material between tissue and a compression system. The compression system may include a body and a plurality of engagement members extending from the body. The engagement members may be adapted to at least partially traverse the material and may include: a support at least partially traversing the material; at least one securing member extending from the support and adapted to engage the tissue. The method may further include urging the compression system toward the tissue such that the body forces the material into contact with the tissue and the securing members engage the tissue and prevent separation of the body and the tissue.

Also disclosed herein in various embodiments is a system for securing an associated material to associated tissue, comprising: an engagement member, the engagement member comprising: a first support having a length for traversing the associated material; a first securing member extending from the first support and adapted to engage the associated tissue; and a retaining member operatively coupled to the first support, for preventing the entire engagement member from traversing the material.

The he first support and the retaining member may be integrally formed.

In some embodiments, the first securing member has a tip and a base, the base connecting to the first support, and the tip of the first securing member is closer to the retaining member than the base. The first securing member can be an oblique spike extending from the first support at an angle of from about 30 degrees to about 60 degrees relative to a longitudinal axis of the first support. Alternatively, the first securing member can be a curved spike.

In other embodiments, the first support and the retaining member are formed as separate pieces, and the first support and the retaining member are adapted to engage each other such that the distance between the retaining member and the first securing member on the first support is adjustable. For example, the retaining member can comprise a hole with internal threads and a base end of the first support can comprise complementary external threads.

In various embodiments, the engagement member has a plurality of securing members extending from the first support and adapted to engage the associated tissue, the plurality of securing members including the first securing member and a second securing member. The first securing member can be located along the first support further from the retaining member than the second securing member. The first securing member and the second securing member may have different lengths. The securing members can be distributed longitudinally and laterally on the first support.

The engagement member may comprise bioabsorbable material. Sometimes, the engagement member is coated with a wound healing factor comprising at least one of: antibiotics, growth factors, interleukin, topical antibacterial materials, anti-tumor agents, and absorbent materials to control wound drainage.

In particular embodiments, the retaining member is a connector that further comprises at least one additional support operatively coupled to the connector, the at least one additional support also at least one securing member extending therefrom. The connector can be linear, a square grid, an arc, a helix, or a square disk. The first support and the at least one additional support may extend in different directions from the connector. The connector can also include at least one securing member extending directly therefrom.

The system can further comprise a body, the body comprising a substrate and a plurality of holes extending entirely through the substrate, each hole being adapted to receive and hold the engagement member. Each hole of the body may be shaped such that the retaining member remains elevated above a surface of the body, or such that the retaining member enters the body.

Also disclosed herein is a method for compressing a material and a tissue to facilitate wound healing, comprising: placing the material between the tissue and a compression system, the compression system comprising an engagement member, the engagement member comprising: a first support having a length for traversing the material; a first securing member extending from the first support and adapted to engage the associated tissue; and a retaining member operatively coupled to the first support, for preventing the entire engagement member from traversing the material; and urging the compression system toward the tissue such that the material contacts the tissue, the first securing member engages the tissue, and the material and the tissue are compressed together.

The features of the present disclosure will be apparent with reference to the following description and attached drawings. In the description and drawings, exemplary embodiments have been disclosed in detail as being indicative of some of the ways in which the principles of the present disclosure may be employed, but it is understood that the present disclosure is not limited correspondingly in scope. Rather, the present disclosure includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

The following is a brief description of the drawings, which are presented for the purpose of illustrating the exemplary embodiments disclosed herein and not for the purpose of limiting the same.

FIG. 1 illustrates an exemplary embodiment of an engagement member having a support, a plurality of securing members, and a retaining member. The support is a cylinder, and the retaining member is a three-dimensional hexagonal disk. The support and the retaining member are integrally formed.

FIG. 2 illustrates an exemplary embodiment of an engagement member having a shorter length compared to FIG. 1.

FIG. 3 illustrates an exemplary embodiment of an engagement member having a shorter length compared to FIG. 2.

FIG. 4 illustrates an exemplary embodiment of an engagement member having a shorter length compared to FIG. 3.

FIG. 5 illustrates a variation of the support with securing member that can be used in an engagement member. Here, the support is a spike, a securing member protrudes outwardly from the support, and the securing member is a spike.

FIG. 6 illustrates another variation of the support with securing member. Here, the support is a spike, and the securing member is an upwardly rounded (concave) spike.

FIG. 7 illustrates another variation of the support with securing member. Here, the support is a spike, and the securing member is an upwardly angled spike.

FIG. 8 illustrates another variation of the support with securing member. Here, the support is a spike, and the securing member is an upwardly rounded spike

FIG. 9 illustrates another variation of the support with securing member. Here, the support is a spike, and the securing member is an upwardly directed hook with barb.

FIG. 10 illustrates another variation of the support and securing members. Here, the support is a spike, and the securing members are outwardly protruding sawteeth.

FIG. 11 illustrates another variation of the support and securing members. Here, the support is a truncated spike, and the securing members are outwardly protruding sawteeth.

FIG. 12 illustrates another variation of the support with securing member. Here, the support is a spike, and the securing member is an upwardly directed truncated conical surface (or Elizabethan collar).

FIG. 13 illustrates another variation of the support and securing members. Here, the support is a spike, and the securing members are downwardly rounded (convex) spikes.

FIG. 14 illustrates another variation of the support and securing members. Here, the support is a spike, and the securing members are in the form of annular disks.

FIG. 15 illustrates another variation of the support with securing member. Here, the support is a spike, and the securing member is an outwardly protruding helical thread.

FIG. 16 illustrates another variation of the support and securing members. Here, the support is a spike, and the securing members are outwardly protruding hemispherical bumps.

FIG. 17 illustrates another variation of the support and securing members. Here, the support is a truncated spike, and the securing members are barbs.

FIG. 18 illustrates another variation of the support and securing members. Here, the support is a truncated spike, and the securing member is a helical groove.

FIG. 19 illustrates another variation of the support and securing members. Here, the support is a spike, and the securing members are in the form of outwardly protruding planar triangles arranged to form a five-point star at various levels.

FIG. 20 illustrates another variation of the support with securing member. Here, the support is a truncated spike, and the securing member is a jagged barb.

FIG. 21 illustrates another variation of the support and securing members. Here, the support is a spike, and the securing members are outwardly protruding spikes. Compared to FIG. 13, these spikes are curved further towards the support.

FIG. 22 illustrates another variation of the support and securing members. Here, the support is a spike, and the securing members are linear grooves extending outwardly from the support along the longitudinal axis.

FIG. 23 illustrates another variation of the support and securing members. Here, the support is a spike, and the securing members are truncated cylinders.

FIG. 24 illustrates another variation of the support and securing members. Here, the support is a spike, and the securing members are raised areas formed by knurling of the spike.

FIG. 25 illustrates another variation of the support and securing members. Here, the support is a spike, and the securing members are raised areas formed by knurling of the spike. The knurling pattern differs from that of FIG. 24.

FIG. 26 illustrates another variation of the support and securing members. Here, the support is a spike, and the securing members are upwardly angled spikes. These spikes are much thinner (i.e. smaller diameter) than those shown in FIG. 7, and more numerous.

FIG. 27 illustrates another variation of the support and securing members. Here, the support is a four-sided pyramid (all four sides of the same shape and triangular), and the securing members are upwardly angled spikes.

FIG. 28 illustrates another variation of the support and securing members. Here, the support is a four-sided wedge, and the securing members are upwardly angled spikes. Two opposing sides of the wedge are triangular, and the other two opposing sides are rectangular. The spikes are all of the same shape.

FIG. 29 illustrates another variation of the support and securing members. Here, the support is a four-sided wedge, and the securing members are upwardly angled spikes. Two opposing sides of the wedge are triangular, and the other two opposing sides are rectangular. The spikes on the triangular sides are of a different shape from the spikes on the rectangular sides.

FIG. 30 illustrates an exemplary embodiment of an engagement member in which the support and the retaining member are made of separate pieces, and the length of the support can be varied. Here the securing members are arranged in four levels, two securing members at each level and pointing in the same direction.

FIG. 31 illustrates an exemplary embodiment of an engagement member in which the length of the support can be varied. Here, the securing members are arranged at alternating levels and in opposite directions.

FIG. 32 illustrates an exemplary embodiment of an engagement member in which the length of the support can be varied. Here, the securing members are arranged at alternating levels, two securing members per level, and pointing in opposite directions at the same level and alternating directions between adjacent levels.

FIG. 33 illustrates an exemplary embodiment of an engagement member in which the length of the support can be varied. The securing members are arranged in a helical fashion.

FIG. 34 illustrates an exemplary embodiment of an engagement member in which the length of the support can be varied. The securing members are arranged in a helical fashion (more spikes compared to FIG. 33).

FIG. 35 illustrates an exemplary embodiment of an engagement member in which the length of the support can be varied. The securing members are arranged in a helical fashion (more spikes compared to FIG. 34).

FIG. 36 illustrates an exemplary embodiment of an engagement member in which the retaining member is a connector engaging multiple supports, the connector having a linear shape.

FIG. 37 illustrates an exemplary embodiment of an engagement member in which the retaining member is a connector having a square grid shape, with supports protruding from each intersection.

FIG. 38 illustrates an exemplary embodiment of an engagement member in which the retaining member is a connector having an arcuate shape.

FIG. 39 illustrates an exemplary embodiment of an engagement member in which the retaining member is a connector having a helical shape. The supports can extend from the connector in various directions.

FIG. 40 illustrates an exemplary embodiment of an engagement member in which the retaining member is a connector having a linear shape. Here, two securing members extend from the connector. Spikes also extend from the connector itself.

FIG. 41 an exemplary embodiment of an engagement member in which the retaining member is a connector having a three-dimensional square disk shape. The supports extend from the bottom surface of the connector.

FIG. 42 an exemplary embodiment of an engagement member in which the retaining member is a connector having a three-dimensional square disk shape. The supports extend from the four sides of the connector.

FIG. 43 is a perspective view of an exemplary embodiment of a body that can be used with a set of engagement members. The body includes a substrate and a plurality of holes extending entirely through the substrate.

FIG. 44 is a side cross-sectional view showing a body and engagement members being used to compress a material and a tissue together.

FIG. 45 is a side cross-sectional view showing a body and a plurality of engagement members in which the retaining members are elevated above the surface of the body.

FIG. 46 is a side cross-sectional view showing a body and a plurality of engagement members in which the retaining members enter the body, and are not elevated above the surface.

FIG. 47 is a bottom view of an exemplary embodiment of a body and a plurality of engagement members. Here, not all holes in the body are filled, which permits the holes to act as drains.

FIG. 48 is a top view of the body and engagement members of FIG. 47.

### DETAILED DESCRIPTION

A more complete understanding of the components, processes and apparatuses disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. The singular forms “a,” “an,” and “the” include plural referents unless the context clearly dictates otherwise.

As used in the specification and in the claims, the term “comprising” may include the embodiments “consisting of” and “consisting essentially of.”

It should be noted that many of the terms used herein are relative terms. For example, the terms “upward” and “downward” are relative to a center and refer to opposite directions. The terms “upper” and “lower”, or “top” and “bottom” are also relative to a center. Similarly, the terms “inner” and “outer” are relative to a boundary. The terms “inward” and outward” are also relative to a boundary.

The term “disk” is used herein to refer to a three-dimensional solid having two opposite and generally parallel surfaces and at least one side.

The terms “perpendicular” and “parallel” are used herein, and should not be construed as referring to only 90° or 180° relationships. Rather, a ±2° tolerance in either direction is acceptable.

The present disclosure relates to novel systems and methods for facilitating wound healing by improving compressive fixation of materials to tissue. Generally, the system includes (i) an engagement member that comprises (a) a support, (b) one or more securing members, and (c) a retaining member. The system can also include (ii) a body which is used to link one or more engagement members over a larger surface area.

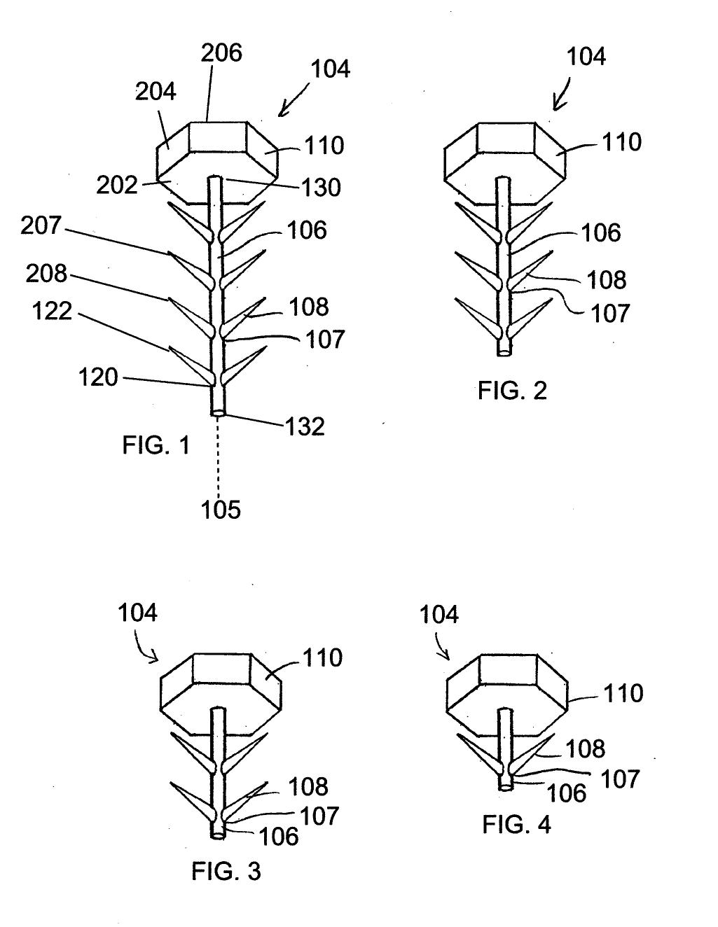

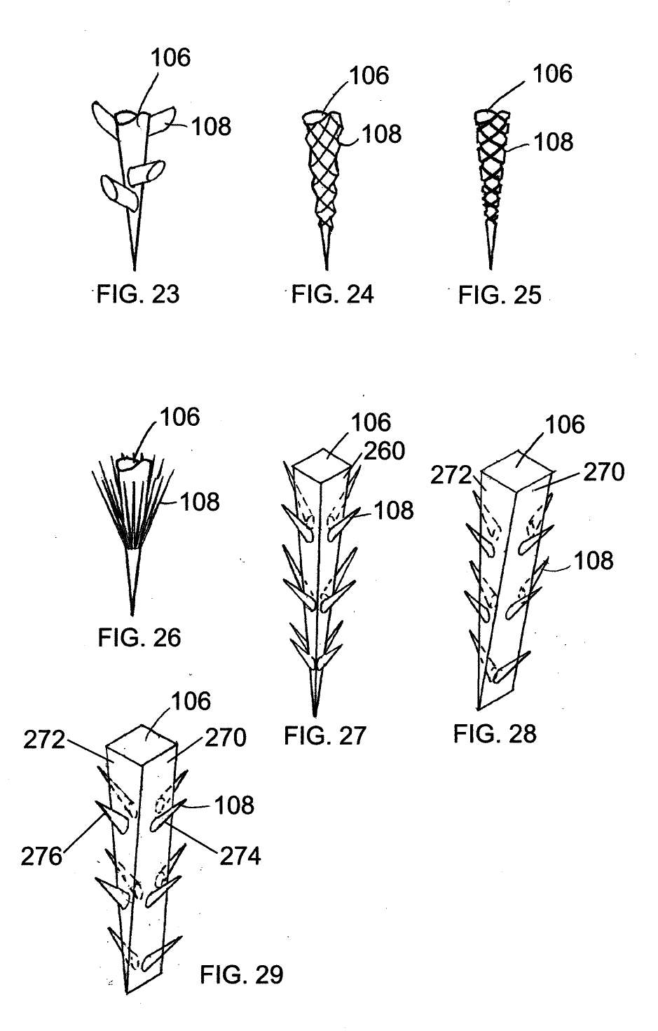

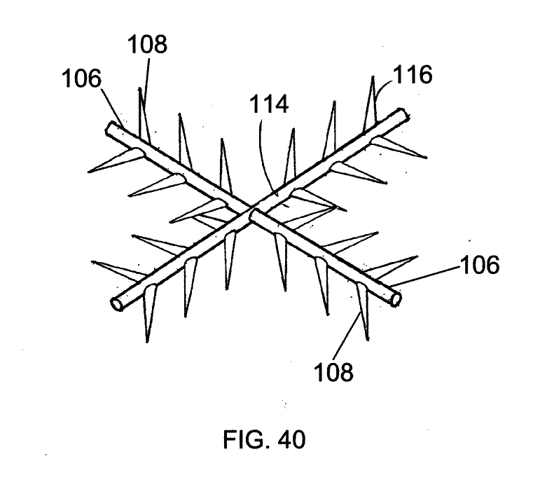

FIGS. 1-48 illustrated several different embodiments of exemplary engagement members 104 adapted to secure and compress a material 102 (e.g., a skin graft), to a tissue 100. One potential use for the present disclosure is in wound healing where it may be useful to maintain pressure to a dressing to facilitate healing by, for example, facilitating the development of blood supply and the incorporation of the material 102 onto the recipient tissue 100. Thus, the engagement member may be useful for providing both pressure and fixation of the material 102 and tissue 100, which in turn may assist vascularization of the material. For the purposes of this disclosure, the term “vascularization” includes vascularization and/or revascularization depending on the circumstances.

Turning specifically now to FIGS. 1-4, the engagement member 104 includes a first support 106, at least one securing member 108 extending from the first support, and a retaining member 110 that is operatively coupled to the first support. Here, the retaining member 110 is a hexagonal disk having a lower surface 202, an upper surface 206, and six sides 204. The first support 106 is coupled to and extends from the lower surface 202. The first support 106 has a base end 130 and a distal end 132, with the base end being adjacent/connected to the retaining member 110.

As depicted here, the first support is a cylinder (i.e. has a cylindrical shape), and eight securing members 108 extend from the first support. The eight securing members are in the shape of oblique spikes and are arranged on four different levels, two spikes per level. Each level is based on the distance of the securing member from the base end 130 of the first support. Here, for example, spike 208 is located further from the retaining member than spike 207.

Each spike 108 has a tip 122 and a base 120, and the base connects to the support 106 at a location 107. Here, the spike 108 is an oblique spike, or has the shape of an oblique cone. In embodiments, the oblique spike extends from the support at an angle of from about 30 degrees to about 60 degrees, measured relative to the longitudinal axis 105 of the support. Here, the spikes are angled upward, or put another way the tip 122 of the spike is closer to the retaining member 110 than the base 120 of the spike.

Generally, the engagement member 104 includes a support 106 operatively coupled to a retaining member 110, wherein the support 106 is adapted to at least partially traverse the material 102. The support may be pointed or have a blunt or otherwise flat tip. For example, the support 106 may be generally shaped like a spike, or may have a generally cylindrical shape, or may be multifaceted (having a plurality of sides). Also, the facets of the support 106 may extend the entire length of the support 106, or only over part of the support 106. For example, facets may exist only on the bottom portion of the support 106. Alternatively, the facets may exist only on the top portion of the support 106. In addition, extending from the support 106 at one or more locations 107 along the support are one or more securing members 108 adapted to engage the tissue 100. These locations may generally be scattered longitudinally and laterally along the support in a given pattern.

The securing members 108 may be attached to the support 106 or integrally formed with the support 106. In addition, the support 106 may be adapted to being trimmed, such as between locations 107 along the length of the support 106. Thus, the support 106 may be adapted to be trimmed between two securing members 108, such as by standard surgical scissors (e.g., surgical scissors commonly used for cutting suture material), so that the length of the support is shortened and at least one of the plurality of securing members is removed from the support. Preferably, the support 106 is dimensioned and made of suitable material to facilitate trimming with standard surgical scissors. To facilitate trimming, the support 106 may be sufficiently thin such that when the support 106 is trimmed, the base of the support 106 is capable of penetrating the tissue 100 without use of excessive force. This is illustrated in FIGS. 1-4, where the main difference between each figure is the length of the support 106 and the resulting number of levels of spikes. In FIG. 1, there are four levels of spikes (eight total spikes), whereas in FIG. 4 there is only one level of spikes (two total spikes).

The securing members 108 may be any structure (e.g. spikes, tines, barbs, prongs, hooks, spurs, arrowheads, protrusions, etc.) extending from the support that is adapted to engage the tissue 100 and secure the support 106 to the tissue 100 and provide compressive force against the tissue (and the material being applied to the tissue). The securing members 108 may be adapted such that forcing the engagement member 104 toward the tissue 100 is not substantially impeded, but removal of the engagement member 104 from the tissue 100 is impeded due to interaction of the securing members 108 with the tissue 100. The engagement member 104 may also be adapted to facilitate compression of the material 102 and the tissue 100. In addition, compression of the material 102 and the tissue 100 may facilitate vascularization of the material 102, which may be useful in skin graft applications where the material 102 is a skin graft.

The shape of the securing members 108 may be varied depending, for example, on the area or type of tissue 100 and material 102 involved. The securing members 108 may be canted, erect, or curvilinear as desired. The securing members 108 may also be formed of arrowheads or canted hooks, or have a single straight cross-section, or may be formed having any suitable shape for engaging the tissue 100 and providing a compressive force against the tissue. As shown in FIGS. 1-35, the securing members 108 may take a variety of exemplary forms, and it is contemplated that other shapes of securing members 108 may also be used. In addition, at least one of the securing members 108 may extend from the support 106 such that at least part of the securing member 108 is closer to the retaining member 110 than the location 107 where the securing member 108 meets the support 106 is to the retaining member 110 (e.g., having the securing members pointing at an angle toward the retaining member or away from the retaining member). For example, the securing member 108 may be generally curved upwards as it extends from the support 106. In addition, the securing members 108 may extend from the support 106 at an angle (not perpendicular). For example, at least one of the securing members 108 may extend from the support at an angle of from about 30 degrees to about 60 degrees.

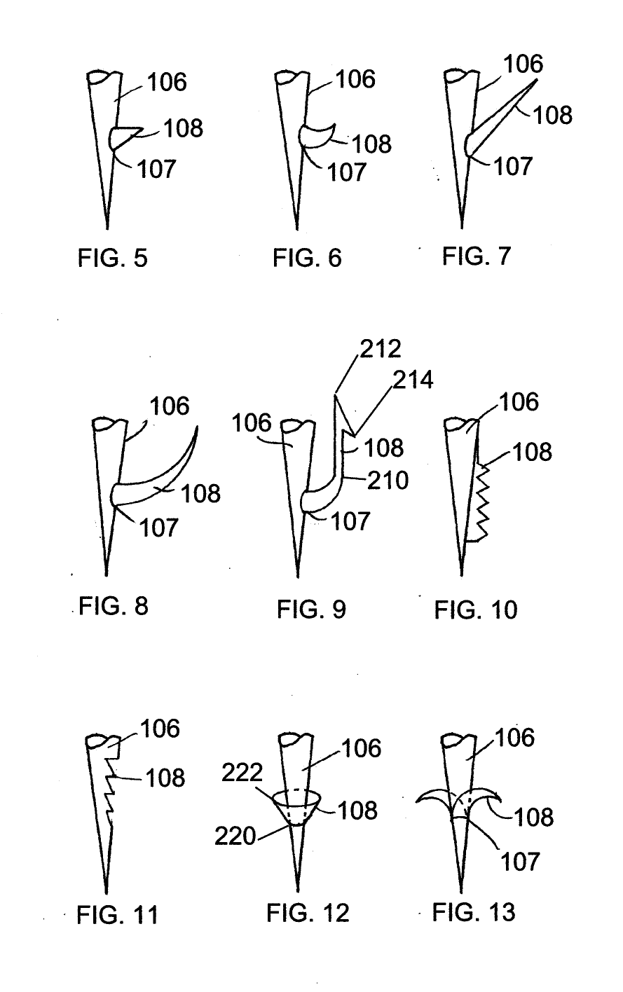

In FIGS. 5-29, several different variations are shown on the shape of the support 106 and the securing member 108. The retaining member is not shown here, but is at the top of each figure. Generally speaking, in many of these figures, the support 106 is in the shape of a spike, or in other words a right circular cone. In some of these figures, the spike is truncated, or put another way is not an entire cone. The present disclosure contemplates that the securing member should be considered as extending from the support. Generally, the support 106 extends perpendicularly from the retaining member 110, though this is not required.

In FIGS. 5-8, the securing member 108 is a spike. In FIG. 5 and FIG. 7, the spike is an oblique spike, and these two figures differ in the length of the spike. In FIG. 6 and FIG. 8, the spike is a rounded or curved spike. In these two figures, the spikes are concave spikes (relative to the retaining member). In contrast, the spikes in FIG. 13 are convex spikes.

The securing member of FIG. 9 is an upwardly pointing hook. The hook includes a shank 210 having a point 212 that extends upward, and a barb 214 that extends downwards.

The securing members of FIG. 10 and FIG. 11 are both sawteeth. They differ in that the sawteeth of FIG. 10 extend from a complete spike, whereas in FIG. 11 they extend from a truncated spike.

The securing member of FIG. 12 is an upwardly directed planar truncated conical surface, or Elizabethan collar. The truncated conical surface has a larger diameter at end 222 compared to the diameter at end 220.

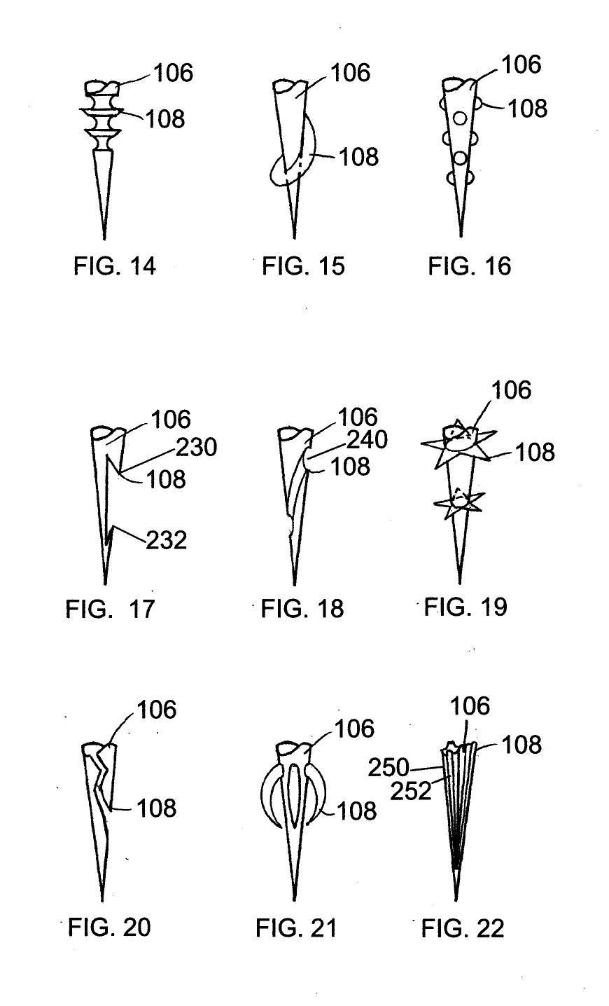

In FIG. 14, the support 106 includes two annular disks 108 extending outwardly and parallel to the retaining member (not visible).

In FIG. 15, the securing member 108 is a helical thread that winds around the support 106. In contrast, the securing member 108 in FIG. 18 is also a helical thread winding around a truncated support 106. The helical thread 108 could be thought of as being created by removing a groove 240 from a complete right circular cone.

In FIG. 17, the securing members 108 include a downward barb 230 an upward barb 232, which extend from a truncated spike 106.

In FIG. 22, the securing members 108 are linear grooves 250 extending from the support 106 along the longitudinal axis. These could be considered as being formed by removing linear grooves 252 from a complete right circular cone.

In FIG. 24 and FIG. 25, the securing members 108 are raised areas formed by knurling the support member 106. The knurling pattern differs between these two figures.

The spikes 108 of FIG. 26 are much thinner in diameter and much longer than the spikes shown in FIG. 1. The spikes in FIG. 26 are all located at the same level.

In FIG. 27, the support 106 is a four-sided pyramid. A pyramid has a polygonal base and triangular sides 260. The spikes 108 extend from the sides of the pyramid.

In FIG. 28 and FIG. 29, the support 106 is a wedge. A wedge has four sides. Two opposite sides 270 are rectangular, and the other two opposite sides 272 are triangular. In FIG. 28, all of the securing members 108 have the same shape. In FIG. 29, the spikes 274 on the rectangular sides 270 are thinner than the spikes 276 on the triangular sides 272.

It is also possible to vary the lengths of the securing members 108 along the support 106, i.e. the securing members have different lengths. For example, it may be desirable for the securing members farthest from the retaining member 110 to be shorter than the securing members 108 closer to the retaining member 110. In such a configuration, the first securing members 108 to engage the tissue 100 may be shorter than the last securing member 108 to engage the tissue, which may facilitate placement of the engagement member 104 and engagement of the securing members 108 with the tissue 100.

In addition, the securing members 108 may be positioned along the support such that they generally extend in all directions from the support 106. For example, they may be placed such that they are approximately 60 degrees from one another around the circumference of the support 106. In this example, the securing members 108 would extend in six different directions from the support 106. Also, the length of the securing members 108 is preferably less than the length of the support 106. For example, the securing members 108 may have a length that is about one tenth the length of the support 106. Moreover, the securing members 108 may also be adapted to be trimmed. Put another way, the securing members can be distributed both longitudinally and laterally on the support.

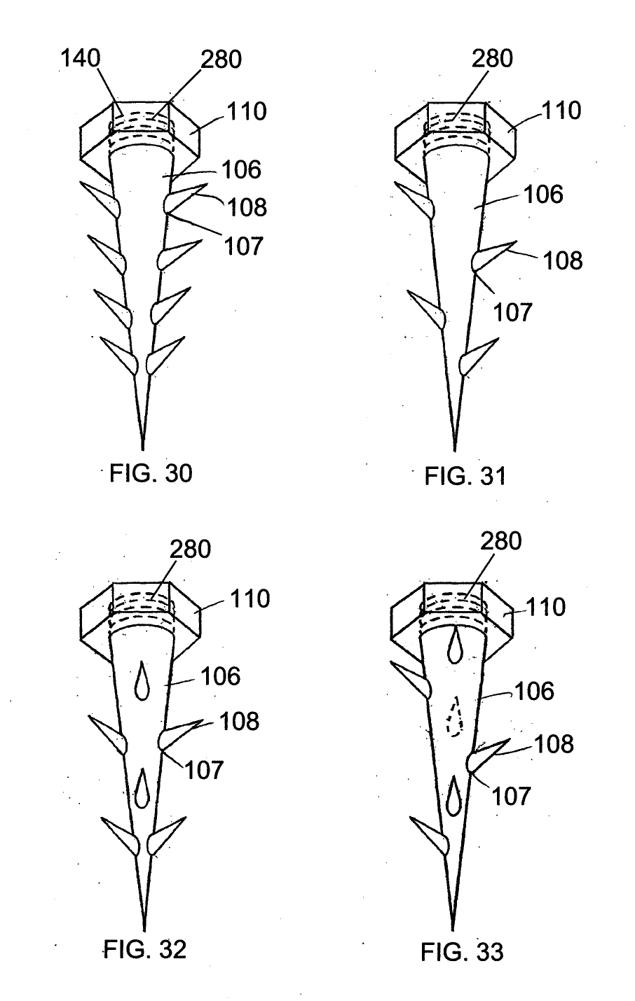

The engagement member 104 may also have a retaining member 110 adapted to prevent the entire engagement member 104 from traversing the material 102. Like the support 106 and the securing members 108, the support 106 and the retaining member 110 may be integrally formed, or the retaining member 110 may be attached to support 106. In addition, as illustrated in FIGS. 30-35, the retaining member 110 may be adjustable such that adjustment of the retaining member changes the distances between the retaining member and the plurality of locations. For example, the support and the retaining member can include cooperative threads, though it will be understood that the retaining member alternatively may be adjusted using any suitable length-adjustment mechanism. The retaining member can generally be of any shape, so long as a transverse or lateral dimension of the retaining member is larger than the transverse or lateral dimension of the support. The lateral dimension of the retaining member is also larger than the extension of the securing member(s) from the support. The retaining member is located at the base end of the securing member.

Referring more specifically now to FIGS. 30-35, in some embodiments the retaining member 110 and the support 106 are formed as separate pieces. They are shaped such that they can engage each other. The retaining member 110 includes a hole 140 with internal threads. The base end 280 of the support includes complementary external threads, and the threads can be used to adjust the length of the support member. Put another way, the distance between the retaining member and any given securing member 108 can be adjusted.

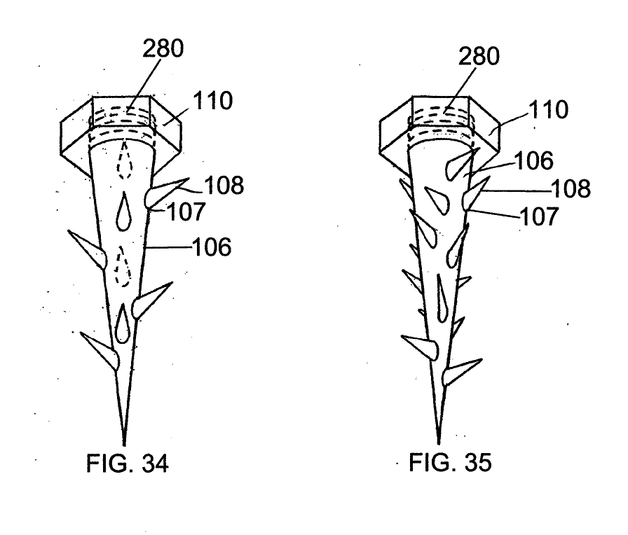

Different methods of positioning the securing members can also be seen in FIGS. 30-35. In FIG. 30, the securing members 108 are placed in four different levels. Each level has two spikes, and the two spikes on each level are located 180 degrees from each other around the circumference of the support. In FIG. 31, there are four levels, but only one spike on each level. Adjacent levels are located 180 degrees from each other around the circumference of the support. In FIG. 32, there are four different levels and two spikes in each level. The two spikes on the same level are located 180 degrees from each other around the circumference of the support. Spikes on adjacent levels are located 90 degrees from each other. In FIG. 33 and FIG. 34, the spikes wind helically around the support 106. In FIG. 35, the spikes are located randomly about the spike.

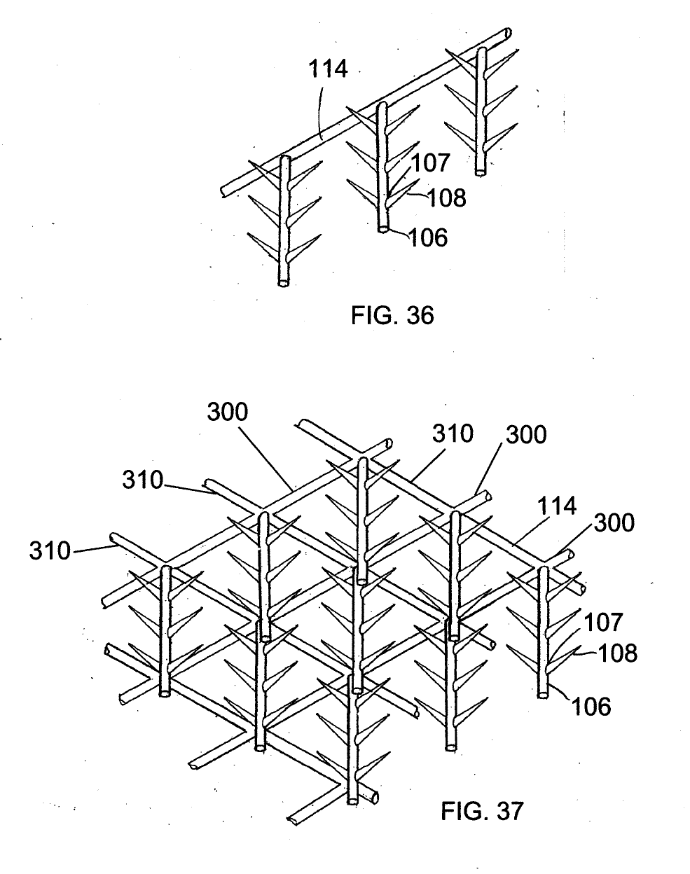

It is also contemplated that in some embodiments, the engagement member 104 has a retaining member that operates as a connector 114 joining multiple supports 106, as shown in FIGS. 36-42. In such embodiments, it should be understood that the connector 114 is also considered the retaining member previously described.

In FIG. 36, the connector 114 is linear, and three securing members 106 extend from the connector. The three securing members all extend in the same direction.

In FIG. 37, the connector 114 is formed from longitudinal members 300 and lateral members 310, which together form a square grid. Securing members 106 are located at the intersections of the longitudinal members 300 and the lateral members 310 (though it should be noted, not at all intersections). The securing members all extend in the same direction, or put another way are all parallel to each other.

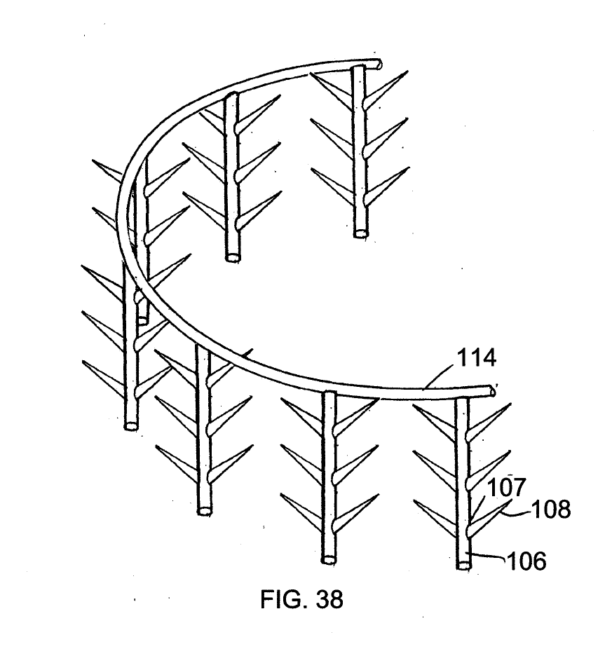

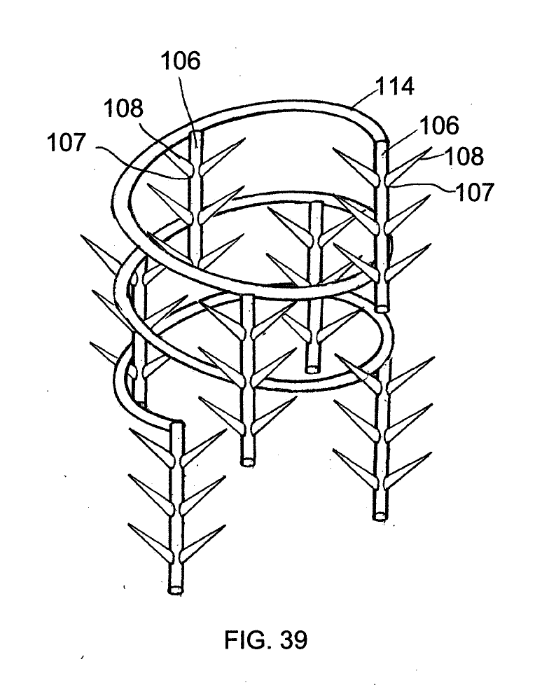

In FIG. 38, the connector 114 is an arc, i.e. has an arcuate shape. In FIG. 39, the connector 114 is a helix. The helix illustrated here is a circular helix, although conical helices are also contemplated. The helix can be right-handed or left-handed.

In FIG. 39, the connector 114 is linear. Two support members 106 extend from the connector 114, and extend in opposite directions from each other. Here, the connector 114 also has securing members 116 extending directly from the connector itself.

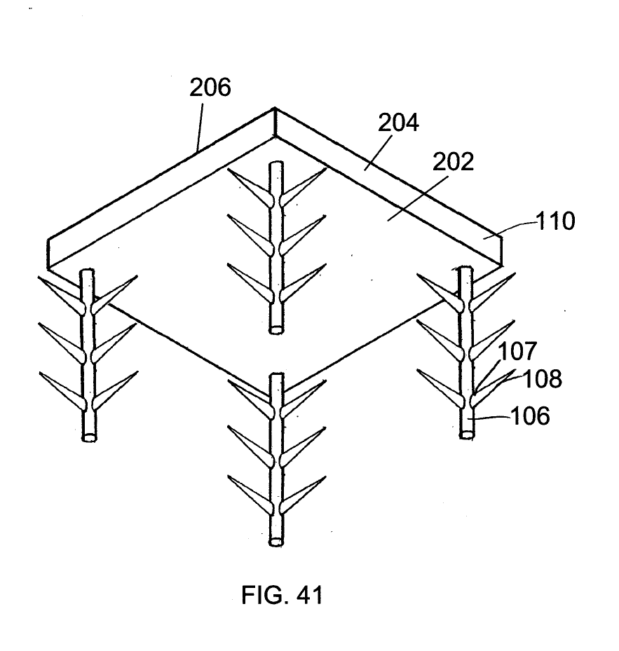

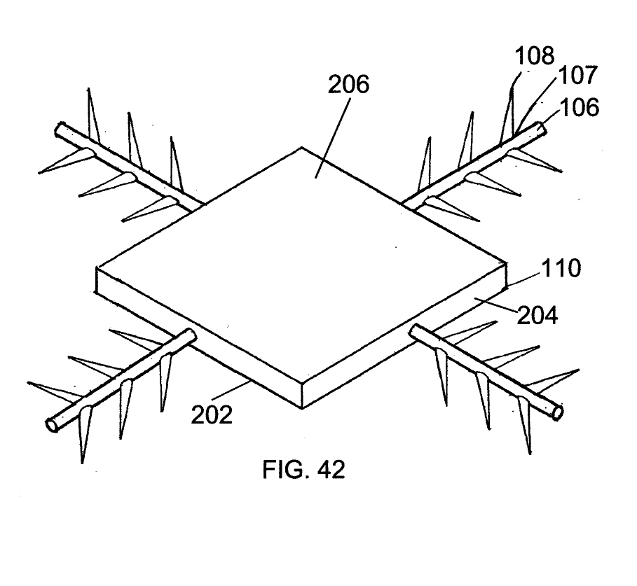

In FIG. 41 and FIG. 42, the retaining member 110 is a square disk with a lower surface 202, an upper surface 206, and four sides 204. In FIG. 41, the four supports 106 extend from the lower surface 202. In FIG. 42, a support 106 extends from each side 204 of the disk 110.

The support 106 and/or securing members 108 may be adapted to interact with tissue 100 that is biologic or synthetic, or a combination of biologic and synthetic. For example, the securing members 108 may be adapted to engage tissue 100 that is biologic or synthetic, or a combination of biologic and synthetic.

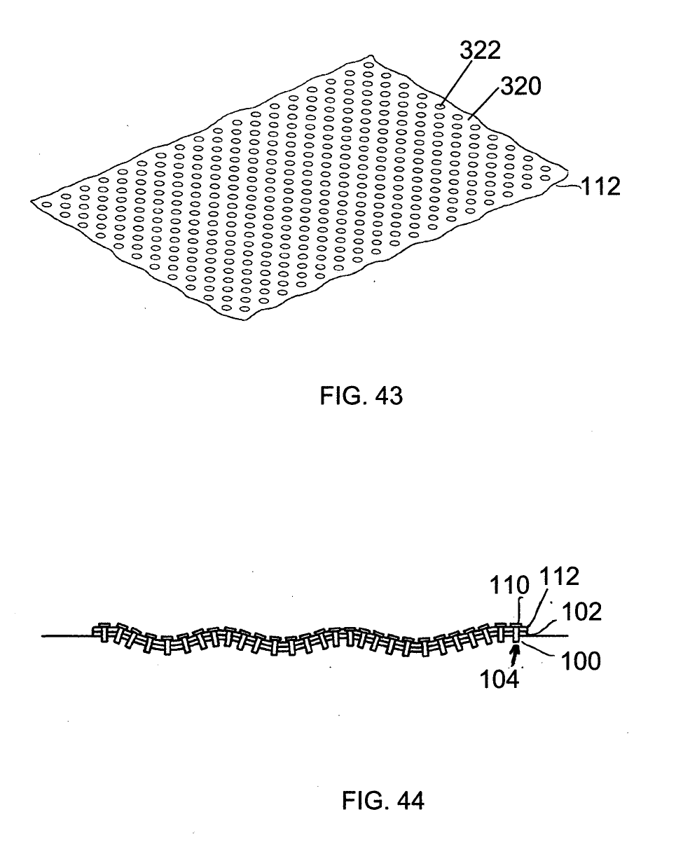

In addition, multiple engagement members 104 may be used, and the engagement members 104 may have supports 106 of different lengths. Turning now to FIGS. 43-48, these illustrate different embodiments of systems comprising a body and engagement members. Generally, the system may include a body 112 and a plurality of engagement members 104 extending from or through the body 112.

Turning first to FIG. 43, the body 112 includes holes that are used to both engage the engagement members and to facilitate drainage from the tissue 100. The body 112 may be pliable and may have the form of a sheet. The specific pliability of 112 may vary depending on the use. For example, it may be desirable for the body 112 to be pliable when used in wound healing over a large area over a surface that is not generally flat. When used with a generally flat surface, pliability may be less desirable. Here, the body 112 comprises a substrate 320 and holes 322 that extend entirely through the substrate.

As seen best in FIG. 44, the body 112 and a plurality of engagement members 104 may be used to compress a material 102, which may be, for example, a graft, and a tissue 100. The engagement members 104 are placed so that they extend through the material 102 and into the tissue 100. As the body 112 is urged toward the tissue 100, the material 102 is compressed onto the tissue 100.

The engagement members 104 include securing members 108 that can engage the tissue 100, thereby impeding the separation of the body 112 and the tissue 100, which in turn, may maintain compression of the material 102 and the tissue 100. Compression of the material 102 and the tissue 100 may facilitate vascularization of the material. In addition, each of the engagement members 104 may include an adjustable retaining member 110 that facilitates adjustment of the pressure of the material body 112, and thus the material 102, on the tissue 100. In addition, each of the engagement members 104 may also be adjustable in length, such as by trimming the support 106 of the engagement member 104. The engagement member 104 may be separate from the body 112 and be used as needed depending on the geometry of the wound. Each engagement member 104 can be trimmed and adjusted based on the needed depth of penetration. While the body 112 is shown with engagement members 104 having retaining members 110, it will be appreciated that the body 112 may serve as a large retaining member with supports 106 and engagement members 108 extending from the body 112 (e.g., extending from the body around the perimeter of the body).

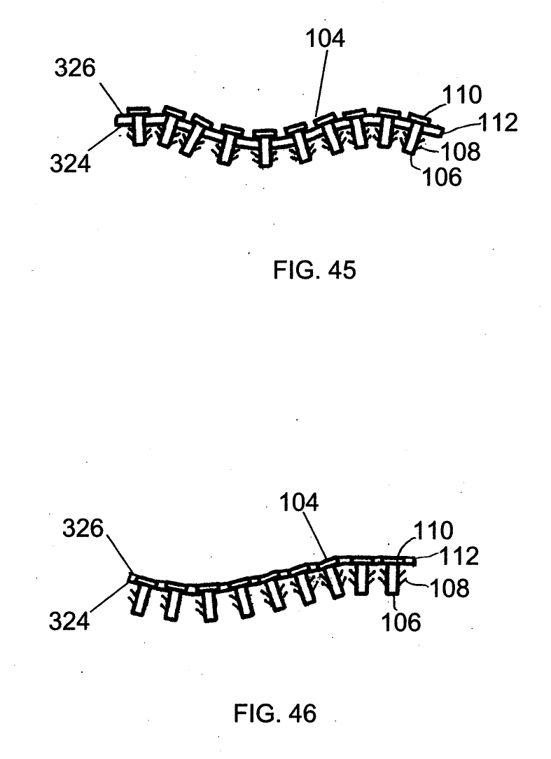

FIG. 45 is a side cross-sectional view of the body 112 and engagement members 104. The body 112 has an upper surface 326 and a lower surface 324. In this embodiment, the retaining member 110 is wider than the hole in the body. As a result, the retaining member is elevated above the upper surface 326, and only the support 106 extends through the body.

In contrast, in FIG. 46, the hole in the body is sized to accommodate the retaining member 110, and the retaining member thus enters the body 112, or put another way the retaining member 110 is located wholly between the upper surface 326 and the lower surface 324.

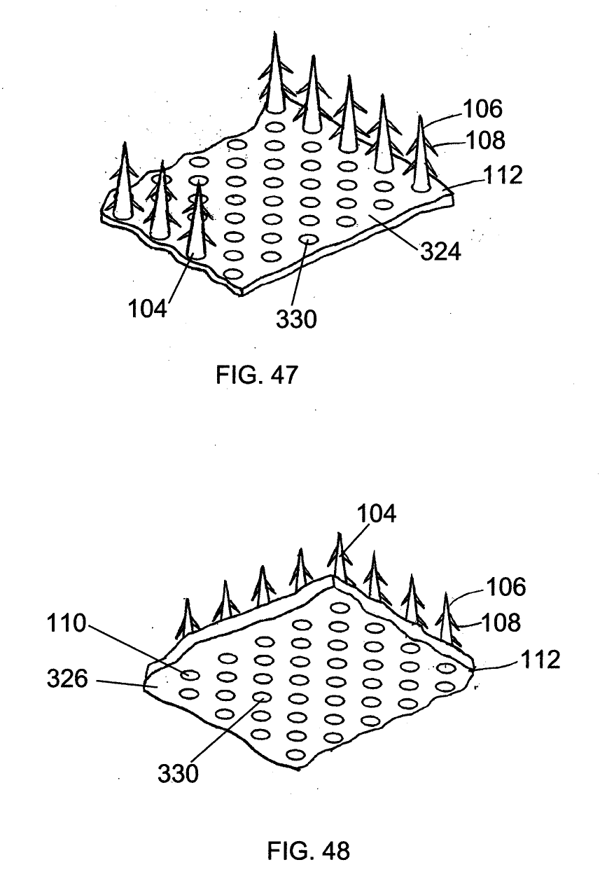

FIG. 47 and FIG. 48 are two perspective views showing another embodiment of the system using a body 112 and multiple engagement members 104. As seen here, there are multiple engagement members 104 extending through the holes of the body 112. There are also empty holes 330 remaining, which can act as pores for drainage of fluids from the wound being covered. The supports 106 extend from the lower surface 324 of the body 112. The retaining members 110 are located wholly within the body 112, and are not elevated above the upper surface 326.

At least part of the engagement member 104 and/or body 112 may be made from bioabsorbable material, such as that used for suture materials. Examples of suitable materials may include polyglycolide, polylactide, poly-α-caprolactone, polydiaxanone, polyglyconate, polylactide-co-glycolide, block and random copolymers of these polymers, copolymers of glycolic, lactic, and other α-hydroxy acids. In addition, an engagement member 104 may be made of two or more types of polymers or copolymers (or molecular weights of the same polymer or copolymer). For instance, the support 106 material might be produced from a more flexible polymer and the securing members 108 of a stiffer material, and the body 112 may be produced from a more flexible material than the support 106.

In addition, the support 106 may be similar in length and width to a prong of a staple. For example, the support 106 may have a length of from about 3 millimeters to about 15 millimeters and may have a width of from about 1 millimeter to about 5 millimeters. Depending on the material used, the support 106 may have a width similar to that of a suture. It will be understood by those of skill in the art that the size and shape of the engagement member 104 may vary depending on the particular application and type of tissue, just as the material of the engagement member 104 may vary. The engagement members 108 may also vary in dimension and material. For example, the engagement members 108 may have a length of from about 1 millimeter to about 5 millimeters and may have a width of from about 0.5 millimeters to about 4 millimeters.

Part of the engagement member 104 and/or body 102 may also be made from less absorbable materials, such as cobalt-chromium alloys, ceramics (alumina ceramic, zirconia ceramic, yttria zirconia ceramic, etc.), titanium, ultra high molecular weight polyethylene (UHMWPE), pyrolytic carbon, titanium/aluminum/vanadium (Ti/Al/N) alloys, Tantalum, carbon composite materials and combinations thereof.

In addition, at least part of the system, such as the body 112, or the engagement members 104, may be adapted to facilitate delivery of wound healing factors to at least one of the tissue 100 or the material 102. The wound healing factors may include, for example, at least one of: antibiotics, growth factors, interleukin, topical antibacterial materials, anti-tumor agents, and absorbent materials to control the wound drainage.

Various methods may be utilized without departing from the scope of the present disclosure. For example, the one method for compressing a material 102 and a tissue 100 to facilitate wound healing may include placing a plurality of engagement members 104 at least partially through the material 102, where the engagement members 104 include a support 106 at least partially traversing the material 102. The engagement members 104 may further include at least one securing member 108 extending from the support 106 and adapted to engage the tissue 100, as well as a retaining member 110 adapted to prevent the engagement member 104 from completely traversing the material 102. The method may further include urging the plurality of engagement members 104 into the tissue 100 such that the securing members 108 engage the tissue 100 and prevent separation of the tissue 100 and the material 102.

Another method for compressing a material 102 and a tissue 100 to facilitate wound healing may include placing a material 102 between tissue 100 and a compression system. The compression system may include a body 112 and a plurality of engagement members 104 extending from the body 112. The engagement members 104 may be adapted to at least partially traverse the material 102 and may include: a support 106 at least partially traversing the material 102; at least one securing member 108 extending from the support 106 and adapted to engage the tissue 100. The method may further include urging the compression system toward the tissue 100 such that the body 112 forces the material 102 into contact with the tissue 100 and the securing members 108 engage the tissue 100 and prevent separation of the body 112 and the tissue 100.

While the present disclosure has been described in association with exemplary embodiments, the described embodiments are to be considered in all respects as illustrative and not restrictive. Such other features, aspects, variations, modifications, and substitution of equivalents may be made without departing from the spirit and scope of this present disclosure which is intended to be limited only by the scope of the following claims. Also, it will be appreciated that features and parts illustrated in one embodiment may be used, or may be applicable, in the same or in a similar way in other embodiments.

Although the present disclosure has been shown and described with respect to certain embodiments, it is obvious that certain equivalents and modifications may be apparent to those skilled in the art upon the reading and understanding of the specification. The present disclosure includes all such equivalents and modifications, and is limited only by the scope of the following claims.

What is claimed is:

1. A system for securing an associated material to associated tissue, comprising:
an engagement member, the engagement member comprising:
a first support having a length for traversing the associated material;
a first securing member extending from the first support and adapted to engage the associated tissue; and
a retaining member operatively coupled to the first support, for preventing the entire engagement member from traversing the material.

2. The system of claim 1, wherein the first support and the retaining member are integrally formed.

3. The system of claim 1, wherein the first securing member has a tip and a base, the base connecting to the first support, and the tip of the first securing member is closer to the retaining member than the base.

4. The system of claim 3, wherein the first securing member is an oblique spike extending from the first support at an angle of from about 30 degrees to about 60 degrees relative to a longitudinal axis of the first support.

5. The system of claim 3, wherein the first securing member is a curved spike.

6. The system of claim 1, wherein the first support and the retaining member are formed as separate pieces, and the first support and the retaining member are adapted to engage each other such that the distance between the retaining member and the first securing member on the first support is adjustable.

7. The system of claim 6, wherein the retaining member comprises a hole with internal threads and a base end of the first support comprises complementary external threads.

8. The system of claim 1, wherein the engagement member has a plurality of securing members extending from the first support and adapted to engage the associated tissue, the plurality of securing members including the first securing member and a second securing member.

9. The system of claim 8, wherein the first securing member is located along the first support further from the retaining member than the second securing member.

10. The system of claim 8, wherein the first securing member and the second securing member have different lengths.

11. The system of claim 8, wherein the securing members are distributed longitudinally and laterally on the first support.

12. The system of claim 1, wherein the engagement member comprises bioabsorbable material.

13. The system of claim 1, wherein the engagement member is coated with a wound healing factor comprising at least one of: antibiotics, growth factors, interleukin, topical antibacterial materials, anti-tumor agents, and absorbent materials to control wound drainage.

14. The system of claim 1, wherein the retaining member is a connector that further comprises at least one additional support operatively coupled to the connector, the at least one additional support also at least one securing member extending therefrom.

15. The system of claim 14, wherein the connector is linear, a square grid, an arc, a helix, or a square disk.

16. The system of claim 14, wherein the first support and the at least one additional support extend in different directions from the connector.

17. The system of claim 14, wherein the connector also includes at least one securing member extending directly therefrom.

18. The system of claim 1, further comprising a body, the body comprising a substrate and a plurality of holes extending entirely through the substrate, each hole being adapted to receive and hold the engagement member.

19. The system of claim 18, wherein each hole of the body is shaped such that the retaining member remains elevated above a surface of the body, or such that the retaining member enters the body.

20. A method for compressing a material and a tissue to facilitate wound healing, comprising:
placing the material between the tissue and a compression system, the compression system comprising an engagement member, the engagement member comprising:
a first support having a length for traversing the material;
a first securing member extending from the first support and adapted to engage the associated tissue; and
a retaining member operatively coupled to the first support, for preventing the entire engagement member from traversing the material; and
urging the compression system toward the tissue such that the material contacts the tissue, the first securing member engages the tissue, and the material and the tissue are compressed together.
